(19) United States
(12) Patent Application Publication
(10) Pub. No.: US 2009/0208551 A1
(43) Pub. Date: Aug. 20, 2009

(54) Biological implantation material and method for preparing same

(75) Inventors: In Seop Kim, Daejeon (KR); Dae Gu Son, Daegu (KR); Young Chul Jang, Seoul (KR); Eun Kyung Yang, Chungcheongbuk-do (KR); Sung Po Kim, Chungcheongbuk-do (KR); Jong Myoung Hong, Chungcheongbuk-do (KR); Ji Hoon Joo, Chungcheongbuk-do (KR); Jong Sang Kim, Chungcheongbuk-do (KR); Sam Hyun Jung, Chungcheongbuk-do (KR); Jong Won Lee, Chungcheongbuk-do (KR); Mi Young Kwon, Chungcheongbuk-do (KR)

Correspondence Address: Baker & Hostetler LLP;Attn: Jim Coffman, 45 Rockefeller Plaza, New York, NY, 10111, US

(73) Assignee: BIOLAND LTD., Cheonan-si (KR)

(21) Appl. No.: 12/217,995

(22) Filed: Jul. 10, 2008

(30) Foreign Application Priority Data
Feb. 14, 2008 (KR) 10-2008-0013379

(51) Int. Cl. A61K 9/70; A61P 43/00

(52) U.S. Cl. 424/423; 424/422; 424/427; 424/430; 424/437; 424/434

(57) ABSTRACT

The present invention relates to a biological implantation material and method of preparing the same, which comprises the steps of:

(i) treating a tissue derived from animal or human with alcohol;

(ii) contacting the said tissue with an enzyme selected from the group consisting of dispase, DNAse, RNAse and pepsin in a solvent;

(iii) treating the tissue obtained in step (ii) with alkaline solution; and

(iv) treating the tissue obtained in step (iii) with acid solution.

### FIELD OF THE INVENTION

The present invention relates to a biological implantation material and method for preparing the same.

### BACKGROUND OF THE INVENTION

Biological implantation material which is implantable medical prothesis and an artificial tissue to the defective tissue or organs by treating the tissue derived from animal and human with chemicals comprises substitute of heart valve, blood, ligament, and cerebral meninges and a wound dressing for treating sun burn, which are.

Skin is the principal organ in the body, which prevents an outflow of body fluid, protects the body from exterior noxious substances such as bacterium and performs thermoregulation. Provided the skin is damaged by sun bum, a body fluid outflows, an infection occurs by dermis exposed to exterior noxious substances therefore the defective skin must be protected from exterior circumstances as soon as possible. Accordingly, the wound dressing used for protecting the defective tissue must have functions, which block the exterior noxious substances and protect the defective tissue while maintaing a suitable permeability of water.

Genenally, synthetic macromolecular materials such as urethane polymer and poly-L-leucine polymer are widely used as a material of the wound dressing. However, synthetic macromolecular materials merely substitute the body tissue with foreign substance due to lack of biological functions thereof. Therefore, many studies for a method of preparing a novel biological material for human implantation, which has a bioaffinity and a biocompatibility by using tissue-derived material have been conventionally developed. For example, studies that biological material for human implantation utilizing bovine amnion effects on treating sun bum by reducing inflammations, and improving healings and is utilized as a wound dressing, and a substitute for reconstruction of defective urinary bladder tissue are reported.

However, for clinical use of the biological material for human implantation utilizing bovine amnion, immunogenic components oriented from bovine have to be removed and viruses oriented from bovine are removed to ensure safety.

An article of commercial, a biological material for human implantation used as a wound dressing and a substitute for reconstruction of defective soft tissues which is prepared by removing immunogenic components from porcine inferior small intestine mucosa and inactivating viruses using peracetic acid, is relatively widely used. However, this material has a short durability by in vivo calcification.

In addition, U.S. Publication Patent No. 2006/0024380 to Ginger A. Abraham discloses a method to remove immunogenic components treating acids, alkali solution, chelating agents and salts. However, the process induces a modification of protein such as collagen due to a treatment of alkali solution having an excessive concentration (pH 12) and has a difficulty in removing cells included in a complex structure such as a substance layer due to enzyme untreatment for removal of cellular matrix.

Therefore, an improved biological material for human implantation prepared by removing immunogenic components completely to prevent an inflammatory response, and an infection from infectious cause such as virus and inhibiting in vivo calcification despite a long-term implantion has been currently required.

Accordingly, the present inventors developed a dermis substitute prepared by using amnion and collagen sponges, which represents wound healing effects (see, Korea Patent No. 644078) and also have attempted to develop an improved method for preparing a biological material for human implantation characterized in inactivating infectious cause such as virus, inhibiting in vivo calcification and having a biocompatibility.

### SUMMARY OF THE INVENTION

Accordingly, it is an object of the present invention to provide a biological implantation material and method for preparing the same.

In accordance with one aspect of the present invention, there is provided a biological implantation material and method of preparing the same, which comprises the steps of:

(i) treating a tissue derived from animal or human with alcohol;

(ii) contacting the said tissue with an enzyme selected from the group consisting of dispase, DNAse, RNAse and pepsin in a solvent;

(iii) treating the tissue obtained in step (ii) with alkaline solution; and

(iv) treating the tissue obtained in step (iii) with acid solution.

### BRIEF DESCRIPTION OF THE DRAWINGS

The above and other objects and features of the present invention will become apparent from the following description of the invention taken in conjunction with the following accompanying drawings, which respectively show:

FIG. 1a is a masson trichorome stain photomicrograph of a bovine amnion tissue;

FIG. 1b is a masson trichorome stain photomicrograph of an amnion implantation material prepared in Example 1;

FIGS. 2a and 2b are a haematoxylin and eosin stain (H&E) photomicrograph of guinea pig tissue applied with an amnion implantation material prepared in Example 1 at 2 weeks and 4 weeks after an application, respectively;

FIGS. 3a and 3b are a H&E stain photomicrograph of guinea pig tissue applied with Surgisis™ at 2 weeks and 4 weeks after an application, respectively;

FIG. 4a is eyes of canine model applied with a filter paper steeped with 1N NaOH;

FIG. 4b is a cornea of canine model modified by alkali burn;

FIG. 4c is a canine model removing the residual NaOH from canine eyes with normal saline;

FIG. 5a is a H&E stain photomicrograph of the tissue modified by alkali bum without any treatments at 6 days after an application; and

FIG. 5b is a H&E stain photomicrograph of the tissue modified by alkali bum applied with an reinforced amnion implantation material prepared in Example 6 at 6 days after application.

### DETAILED DESCRIPTION OF THE INVENTION

In step (i), alcohol is treated to a tissue derived from animal or human so as to remove lipids, inactivate viruses and inhibite in vivo calcification.

The tissue may be cardiac valve, inferior small intestine mucosa, ligament, blood vessel, skin, bone, fascia and amnion derived from animal or human, preferably bovine fascia and amnion, porcine aortic valve, small intestine and heart valve, more preferably bovine amnion.

The alcohol can be treated to the said tissue in an amount of ranging from 80 to 95% (preferably, 95%) volume/volume and storaged for at least 12 hours at 4 to 10° C. so as to remove lipids from the tissue (the first treatment). Thereafter, the alcohol can be retreated to the tissue in an amount of ranging from 40 to 80% (preferably, 70%) volume/volume and storaged for at least 12 hours at 4 to 10° C. to inhibit causative agents of in vivo calcification and inactivate viruses (the second treatment).

In step (ii), the tissue obtained in step (i) is contacted with an enzyme in a solvent so as to remove alkaline components and residual lipids.

The enzyme may be selected from the group consisting of trypsin, dispase, DNAse, RNAse and pepsin, preferably trypsin in an amount of ranging from 0.02 to 0.2% weight/volume. The solvent used in this reaction may further comprise 0.01 to 0.5% of ethylenediamine tetraacetic acid (EDTA) and 0.05 to 5% of sodium chloride, preferably and be subjected to an enzyme reaction at a temperature ranging from 25° C. to 40° C. (preferably, 37° C.) for 10 mins to 2 hours (preferably, 1 hour).

Further, the tissue obtained in step (i) may be treated with a solvent whose pH is ranging from 9.0 to 11.4, comprising 0.01 to 2% of EDTA and 0.05 to 5% of sodium chloride prior to conducting the step (ii) to remove soluble alkaline impurities.

In step (iii), the tissue obtained in step (ii) is treated with alkaline solution to remove immunogenic components.

The alkaline solution may comprise EDTA and sodium chloride, whose pH is ranging from 9.0 to 11.4, preferably 11.0.

In step (iv), the tissue obtained in step (iii) is treated with acid solution.

The acid solution may comprise 0.02 to 2% of EDTA or hydrochloric acid, whose pH is ranging from 1.7 to 2.3, preferably 2.

In step (iii) or (iv), sodium hydroxide having at least 11.5 of pH concentration or hydrochloric acid having less than 1.7 of pH concentration may cause a modification of the tissue.

Furthermore, a reinforced biological implantation material that physical and mechnical intensity is more reinforced than the biological implantation material prepared in step (iv) can be prepared by placing at least 2 sheets of the biological implantation material obtained in step (iv) between 2 molds, attaching the sheets to the mold and subjecting to a freeze drying and a crosslinking reaction. Specifically, the reinforced biological implantation material can be prepared by placing at least 2 sheets of the biological implantation material obtained in step (iv) between 2 molds which have a pore of a thickness ranging from 1 to 10 cm and made of copper or aluminium; pressing to the molds under a pressure ranging from 1 to 20 mb; and subjecting to a freeze drying at a temperature ranging from −20 to −130° C. (preferably, −40° C.) for 4 to 72 hours (preferably, 18 hours) and a conventional crosslinking reaction.

The conventional crosslinking reaction may be conducted by treating with 0.25% of glutaraldehyde (GAD), treating with a mixture of 33 mM of 1,3-carbodiimide and 6 mM of N-hydroxysuccinimide to 90% of acetone, treating with a mixture of 33 mM of 1,3-carbodiimide and 6 mM of N-hydroxysuccinimide to 40% of alcohol or UV crosslinking and dehydrothermal (DHT) crosslinking.

A method for preparing a biological material by a laminar flow drying disclosed in U.S. Patent Publication No. 2003/0130747 to Ginger A. Abraham et al. may cause a contraction of the tissue due to surface tension between the tissue and water molecule induced by water evaporation in tissue. In contrast to the method of the present invention by the freeze drying is able to prevent the modification of the tissue and consist of a desired regular form.

In addition, a method for preparing a biological material by inserting collagen or gelatin-coated mesh between amnions disclosed in U.S. Pat. No. 5,876,451 to Tooru Yui et al. may cause an overall increased mechanical intensity through a complementation of thickness, but it may cause a declined long-term endurance due to a weak binding strength between the tissue and mesh. In contrast to the method the present invention by the freeze drying using molds is able to increase a long-term endurance induced by an increased togetherness among tissues.

The biological implantation material according to the present invention is characterized in that:

(a) there is provided an entire substrate that an alive epithelium, endothelium and nerve cells are attachable;

(b) there is no in vivo immunorejection following the implantation;

(c) there is no in vivo calcification following the implantation;

(d) the percentage of collagen is calculated to be at least 95%; and

(e) biological implantation material prepared may be used as wound dressing, substitute for corneal epithelium, implant for reinforcing soft tissue, implant for reconstructing peritoneum, substitute for meninges, substitute for ear drum, substitute for reconstructing urinary bladder, adhesion protective agent or implant for treating urinary incontinence.

The following Examples are intended to further illustrate the present invention without limiting its scope.

### EXAMPLE 1

### Preparation of Amnion Implantation Material

Bovine amnion samples collected from a bovine placenta were storaged in sterile saline under a cold condition and transported to the laboratory. 500 cm² of the sample collected was treated with 1 L of 95% of ethanol and kept overnight in a cold storage to remove lipids from the bovine amnion sample. The sample was washed three times with 1 L of purified water for 10 mins and removed a substrate layer from the sample using a scrapper. The said sample was storaged in 1 L of 70% of ethanol under a cold condition to inactivate viruses and added 1 L of EDTA/sodium chloride solution (pH 11) comprising 0.2% of ethylenediamine tetraacetic acid (EDTA) and 0.9% of sodium chloride and stirred for 1 hour at 150 rpm to remove soluble alkaline impurities (step (i)). Thereafter, trypsin/EDTA/sodium chloride solution (pH 7.4) comprising 0.05% of trypsin, 0.02% of EDTA, and 0.9% of sodium chloride was treated thereto and subjected to an enzyme reaction while stirring for 1 hour at 37° C. (step (ii)). The amnion sample obtained was treated with 1 L of 70% of ethanol and stirred for 1 hour at 150 rpm to remove residual lipids and the alkaline solution (pH 11) used in step (i) was then treated thereto and stirred for 1 hour at 150 rpm (step (iii)). And acid solution (pH 2) comprising 0.2% of EDTA was then treated thereto and stirred for 1 hour at 150 rpm to swell the resultant amnion sample and washed three times with 1 L of purified water for 30 mins at 150 rpm (step (iv)).

The resultant amnion implantation material of the present invention prepared above may be sterilized by subjecting to a freeze drying or gamma radiation at 25 kGy after packing them, selectively.

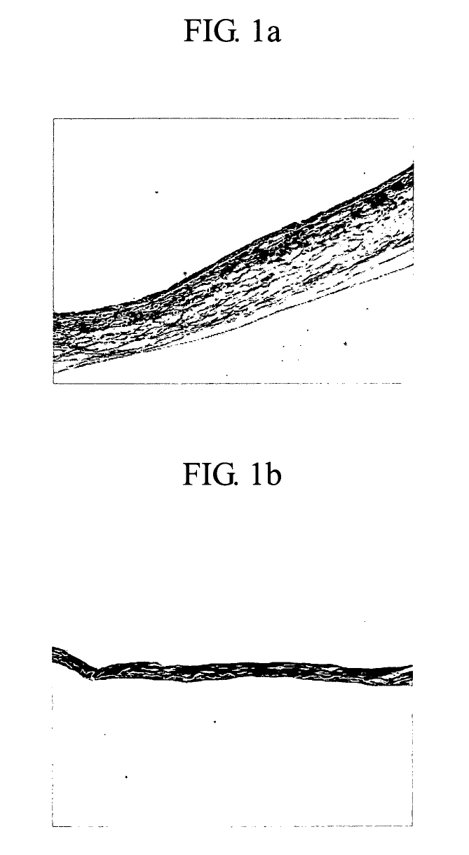

To determine the cell removal histologically in the amnion implantation material prepared above, a masson trichrome staining was performed on both original amnion tissues and treated amnion tissues according to the method of the present invention. The results are shown in FIGS. 1a and 1b, respectively. As shown in FIG. 1b, the treated amnion tissues appeared completely free of epithelial cells present in basilar membrane of amnion and free of cells present in substrate layers compared to the original amnion tissues.

### EXAMPLE 2

### Content of Lipids and Modified Collagens

The efficacy of the method according to Example 1, the method accoding to U.S. Patent Publication No. 2006/0024380 to Ginger A. Abraham et al. (Condition A) and U.S. Pat. No. 5,876,451 to Tooru Yui et al. (Condition B) was determined. The method according to the condition A and B is described in more detail below.

In condition A, the substrates of amnion derived from bovine placenta were removed. The sample was added to 1 L of 0.1 M EDTA/10 mM NaOH solution per 100 cm², stirred for 18 hours at 200 rpm and added to 1L of 1 M HCl/10 mM NaOH solution, stirred for 8 hours at 200 rpm. The resultant sample was treated with 1 L of 1M NaCl/10 mM phosphate buffered saline (PBS), and thereafter stirred for 18 hours, added 1 L of 10 mM PBS thereto and then stirred for 2 hours and further stirred in sterile purified water for 1 hour at 200 rpm.

In condition B, the substrates of amnion derived from bovine placenta were removed. The sample was fully washed with purified water to remove casein-like substrates and 2.5 g of sodium azide, 0.5 g of ficin and 5 L of 0.2 M of PBS solution comprising NaCl in a suitable amount to make 0.9% of concentration thereof (pH 7) were then added thereto and washed fully with pufied water after allowing to stand for 24 hours at a room temperature. And the sample was placed between 2 frames made of propylene, fixed with clips to subject to an ultrasonification for 15 minutes, and thereafter 0.1% of benzalkonium chloride solution was added thereto.

To determine the efficacy of the method according to Example 1, condition A and condition B, the contents of lipids and modificated collagens were determined.

The content measurement of lipids, which cause in vivo calcification was achieved by a sulfo-phospho-vanillin reaction method (see, [J. Microbiological method. 55, 411-418 (2003)]). To each test tube was added 1 mg of each sample, and 2 ml of sulfuric acid and heated to 100° C. and thereafter cooled. 5 mL of phosphoric acid-vanillin was treated thereto, and then stirred for 15 minutes at 37° C. Optical density of the samples treated was determined at 530 nm and the results are shown in Table 1.

TABLE 1
Lipid contents

| | Example 1 | Condition A | Condition B |
|---|---|---|---|
| Lipid contents | 0.04% | 0.24% | 0.25% |

As shown in Table 1, the amnion implantation material according to the present invention represents the lowest lipid contents.

In addition, the content measurement of the modified collagens was achieved by Infra Red (IR) spectroscopy. IR spectroscopy was conductd by applying a reverberatory ac•ces•so•ry ATR to the instrument, and determining a baseline except for an interference. The modified collagen contents were measured in measurement wavelength ranging from 600⁻¹ cm to 1800⁻¹ cm and determined as a relative ratio to peak intensity at 1450⁻¹ cm into peak intensity at 1235⁻¹ cm and the results are shown in Table 2 (see, [I. V. Yannas, J. Macromol. Sci, Rev. Macromol. Chem., 7, 49 (1972)]).

TABLE 2
Modified collagen contents

| | Example 1 | Condition A | Condition B |
|---|---|---|---|
| 1235⁻¹ cm/1450⁻¹ cm | 0.04% | 0.24% | 0.25% |

As shown in Table 2, the amnion implantation material according to the present invention represents the most excellent helical structure of collagens. The modification of collagens was induced by an excessive alkali treatment (pH 12) to the sample in condition A and an ultrasonification to the sample in condition B.

Also, the amnion implantation material prepared in Example 1 represented that the percentage of collagen is calculated to be at least 95% in amino acid analysis by high-performance liquid chromatography (HPLC).

### EXAMPLE 3

### Biocompatibility Test by Hypodermic Implantation to Guinea Pig

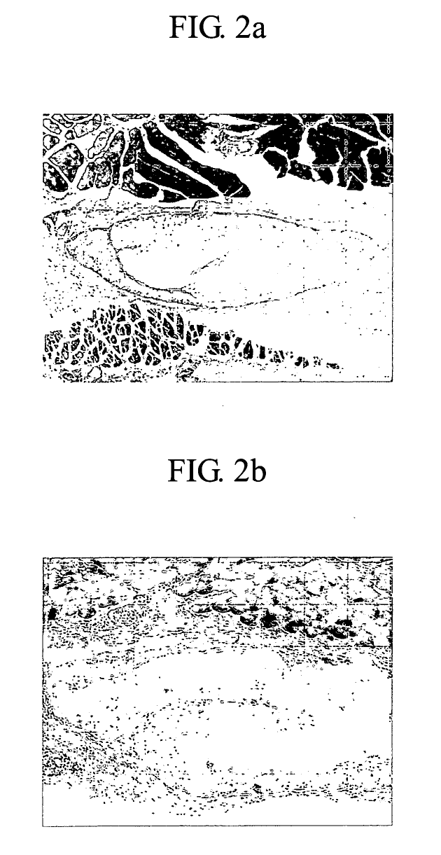

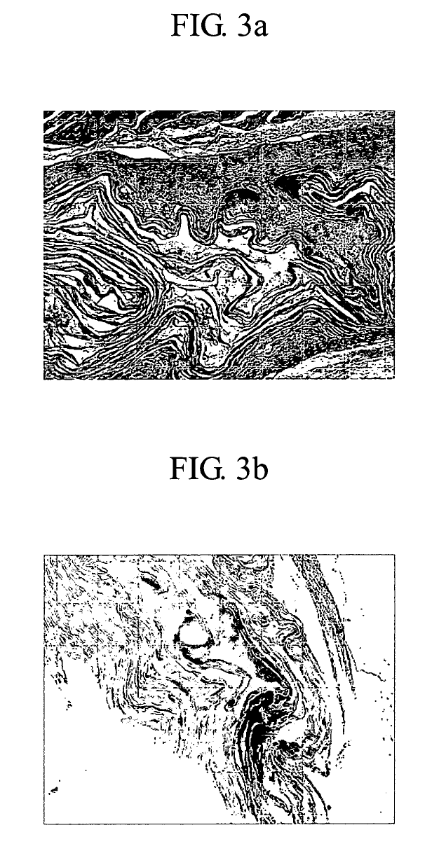

The degree of inflammatory cells and in vivo calcification produced was determined by a hypodermic implantation to guinea pig. The procedure was conducted by comparing a guinea pig tissue which was applied with the amnion implantation material prepared in Example 1 and a guinea pig which was applied with Surgisis™ (Cook Inc. USA) by the hypodermic implantation. 2 weeks and 4 weeks later, the applied tissue was procured from the each guinea pig to fix with formalin, washed and embedded with parapins. The tissue obtained in above cut into 5 μm of thickness, hematotoxyline & eosin (H&E) staining was performed and the stained tissue was then exhibited using optical microscope. After 2 weeks and 4 weeks, H&E stain photomicrograph of the tissue applied with an amnion implantation material prepared in Example 1 was shown in FIGS. 2a and 2b, respectively. Also after 2 weeks and 4 weeks, a H&E stain photomicrograph of the tissue applied with Surgisis™ was shown in FIGS. 3a and 3b, respectively.

As shown in FIG. 2a, a fibroblast infiltration was exhibited in the cells surrounding the tissue applied with the amnion implantation material. In addition, as shown in FIG. 2b, a slow fibroblast infiltration and a new collagen formation was exhibited. Although it passed 4 weeks, an inflammatory response and in vivo calcification was not exhibited, therefore the amnion implantation material of the present invention is biocompatible.

In contrast, as shown in FIG. 3a, a strong lymphocyte infiltration was exhibited in the cells surrounding the tissue applied with Surgisis™ (Cook Inc., USA). Generally, the lymphocytes is related to the immunological reaction, therefore it shows that immunogenic materials are present in the tissue applied with Surgisis™. As shown in FIG. 3b, an immunological reaction as lymphocytes was gretely reduced, but the fibroblastinfiltration was not exhibited and in vivo calcification in numerous regions in the applied tissue was exhibited, therefore it shows that Surgisis™ is not biocompatible as a long-term biological implantation material.

### EXAMPLE 4

### Virus Inactivation Test

To clinically utilize the amnion implantation material according to the present invention, safety from associated viruses of animal-derived tissues must be ensured, therefore the procedure as below was conducted to verify virus inactivation during the method of the present invention according to the requirement of EN12442.

Bovine herpes virus (BHV; ATCC VR-188), Bovine viral diarrhoea virus (BVDV; ATCC VR-534), Parainfluenzavirus 3(PI 3; ATCC VR-281) and Bovine parvovirus (BVP; ATCC VR-767) are selected as a verifying virus to meet the requirement set by FDA and ISO. In treating with 70% of ethanol to the tissue in the step (i) of Example 1, the above each virus storage solution underwent a spiking and each virus inactivation was determined after 1 hour, 6 hours and 12 hours while allowing the each solution to stand at 4° C. As a result, all viruses were completely not discovered and inactivated in the samples treated with 70% of ethanol. Therefore, the procedure of treatment of 70% ethanol in Example 1 is very effective in virus inactivation.

Furthermore, virus inactivation of the amnion material after packing the amnion material within a bag and sterilizing by gamma irradiation was determined. The results are shown in Table 3.

TABLE 3
Virus inactivation test

| | Reduction factor (Log 10) | | | |
|---|---|---|---|---|
| Example 1 | BHV | BVDV | BPV | BPIV-3 |
| Treatment of 70% ethanol | ≧5.29 | ≧4.49 | ≧2.59 | ≧4.81 |
| Gamma irradiation at 25 kGy | ≧6.07 | ≧5.33 | 3.43 | ≧6.29 |
| Log consumption reduction | ≧11.36 | ≧9.82 | ≧6.02 | ≧11.1 |
| factor | | | | |

### EXAMPLE 5

### Structural Protein and Growth Hormone Contents

To determine lost wound healing effective components, quantitative analysis of epidermal growth factor (EGF) and collagen type IV was conducted on contents before treatment and after treatment, respecrively. The quantitative analysis on epidermal growth factor (EGF) and collagen type IV (R&D system Minneapolis, Minn., USA) was conducted by enzyme linked immunosorbent assay (ELISA), which comprises extracting each sample with PBS, centrifuging for 5 minutes at 15,000 rpm, recovering a supernatant therefrom. Further, The quantitative analysis on DNA was determined by dissolving the 25 mg of dried sample in 200 μl of tissue lysis buffer solution using a AccuPrep Genomic DNA extraction kit (Bioneer, Korea) and calculating using a UV photometer.

The results of contents of epidermal growth factor, collagen type IV, and DNA on before treatment and after treatment are shown in Table 4.

TABLE 4
Epidermal growth factor, collagen type IV and DNA contents

| | Contents | Contents |
|---|---|---|
| | before treatment | after treatment |
| EGF (pg/mg) | 1.66 | 0.86 |
| Collagen type IV(pg/mg) | 2.93 | 2.84 |
| DNA (μg/mg) | 6.89 | 0.01 |

As shown in Table 4, the structure of the structural protein such as collagen type IV was well-preserved during the procedure. The contents of epidermal growth factor was lost nearly half, but it still remained massive. The contents of DNA as an immunogenic component was nearly removed during the procedure.

### EXAMPLE 6

### Reinforced Amnion Implantation Material

To reinforce a physical and mechanical intensity of the amnion implantation material prepared in Example 1, the amnion implantation material obtained in step (iii) of Example 1 was placed between 2 alumium molds having at least 5 cm of a pore, and pressed in a sandwich-like form, wherein high-density polyethylene nonwoven was inserted between each aluminum mold and the tissue. And the mold that the tissue was inserted was then freezed in a −40° C. freezer for 18 hours and conducted a freeze drying for 24 hours. Thereafter, dehydrothermal treatment (DHT) crosslinking reaction was performed at 110° C. for 48 hours under a vacumn of 1 mtorr. The reinforced amnion implantation material obtained in above was packed with aluminum packing sheets and then sterilized by gammar irradiation at 25 kGy.

### EXAMPLE 7

### Wound Healing Effect on the Reinforced Amnion Implantation Material

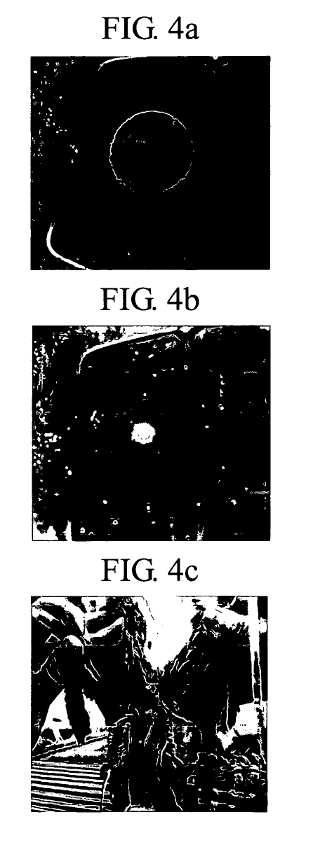

To determine the wound healing effect of the reinforced amnion implantation material on the defective cornea epidermis, eyes of canine model were applied with a filter paper soaked with 1N of NaOH to induce an alkali burn. The picture of canine model applied with 1N of NaOH is shown in FIG. 4a.

After 1 day, the modified cornea epidermises and substances were removed using 8 mm of trephine and a blade and a picture which shows the modified corneas of canine model after removing the filter paper soaked 1N of NaOH from the canine eyes is shown in FIG. 4b. The eyes of canine model that alkali burn was induced were washed with normal saline to remove the residual NaOH therefrom and the picture thereof is shown in FIG. 4c.

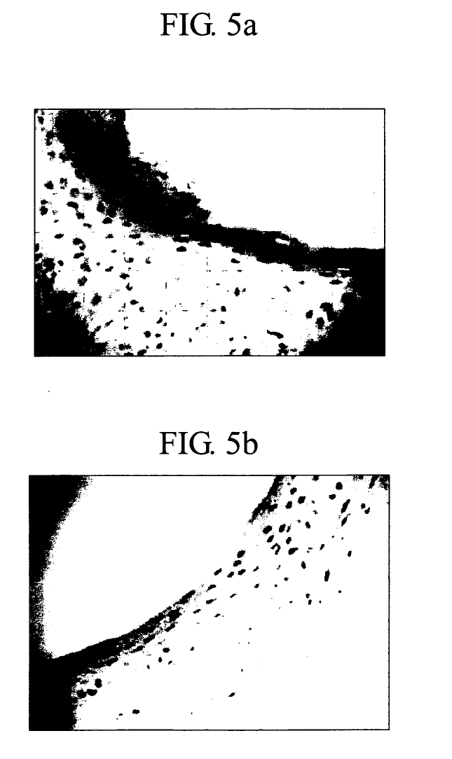

One eye (the right eye) was applied with the reinforced amnion implantation material piece prepared in Example 6, while another eye (the left eye) was allowing to stand without any treatments as a control. After 6 days from the application of the reinforced amnion implantation material piece, the histological analysis was conducted. As a result, the applied right eye exhibited an excellent regeneration of cornea epidermis as shown in FIG. 5a, while the unapplied left eye exhibited an irregular epithelialization, numerous inflammatory cells and fibrosis as shown in FIG. 5b.

While the invention has been described with respect to the above specific embodiments, it should be recognized that various modifications and changes of the invention also fall within the scope of the present invention defined by the claims that follow.

What is claimed is:

1. A method of preparing a biological implantation material, which comprises the steps of:
(i) treating a tissue derived from animal or human with alcohol;
(ii) contacting the said tissue with an enzyme selected from the group consisting of trypsin, dispase, DNAse, RNAse and pepsin in a solvent;
(iii) treating the tissue obtained in step (ii) with alkaline solution; and
(iv) treating the tissue obtained in step (iii) with acid solution.

2. The method of claim 1, wherein step (i) comprises the first treatment of the tissue with alcohol ranging from 80 to 95% volume/volume, and the second treatment of the tissue with alcohol ranging from 40 to 75% volume/volume.

3. The method of claim 1, wherein the enzyme used in step (ii) is trypsin.

4. The method of claim 1, wherein the enzyme concentration is ranging from 0.02 to 0.2% weight/volume.

5. The method of claim 1, the solvent used in step (ii) further comprises 0.01 to 0.5% of ethylene tetraacetic acid (EDTA) and 0.05 to 5% of sodium chloride.

6. The method of claim 1, wherein the tissue obtained in step (i) is treated with a solvent whose pH is ranging from 9.0 to 11.4, comprising 0.01 to 2% of ethylenediamine tetraacetic acid and 0.05 to 5% of sodium chloride prior to performing the step (ii).

7. The method of claim 1, a pH of the alkaline solution is ranging from 10.5 to 11.4.

8. The method of claim 1, a pH of the acid solution is ranging from 1.7 to 2.3.

9. The method of claim 1, the tissue derived from animal or human is selected from the group consisting of pericardium, valvule, inferior small intestine mucosa, ligaments, blood vessel, skin, bone, fascia and amnion.

10. The method of claim 1, wherein the tissue obtained in step (iv) further comprises the step of placing the at least 2 sheets between 2 molds, attaching the tissue to the mold and subjecting a freeze drying and a crosslinking reaction.

11. A biological implantation material prepared according to the method of any one of claims 1 to 10.

12. The biological implantation material of claim 11, wherein the use of the biological implantation material is wound dressing, substitute for corneal epithelium, implant for reinforcing soft tissue, implant for reconstructing peritoneum, substitute for meninges, substitute for ear drum, substitute for reconstructing urinary bladder, adhesion protective agent or implant for treating urinary incontinence.
